(19) United States
(12) Patent Application Publication
(10) Pub. No.: US 2013/0068952 A1
(43) Pub. Date: Mar. 21, 2013

(54) RADIATION DETECTION ELEMENT, RADIOGRAPHIC IMAGE DETECTION PANEL AND RADIOGRAPHIC IMAGING DEVICE

(71) Applicant: Takao KUWABARA, Kanagawa (JP)

(72) Inventor: Takao KUWABARA, Kanagawa (JP)

(73) Assignee: FUJIFILM CORPORATION, Tokyo (JP)

(21) Appl. No.: 13/596,892

(22) Filed: Aug. 28, 2012

(30) Foreign Application Priority Data
Sep. 21, 2011 (JP) 2011-206130

(51) Int. Cl. G01T 1/20; A61B 6/04; G01T 1/00

(52) U.S. Cl. 250/366; 250/394

(57) ABSTRACT

There is provided a radiation detection element including: a radiation detection section that is formed from plural pixels that is of the same size and arrayed two-dimensionally while adjacent to one another, and that detects radiation passed through an object of imaging; plural scan lines that transfer signals that carry out switching control of switching elements provided respectively at the plural pixels; and plural data lines that are disposed so as to intersect the scan lines, and that transfer electric signals read-out by the switching elements, wherein the radiation detection section is made into a shape in which a pair of opposing sides is longer than another pair of opposing sides, and each of the pixels is made into a shape that is short in a short side direction of the radiation detection section and long in a long side direction of the radiation detection section.

### CROSS-REFERENCE TO RELATED APPLICATION

This application is based on and claims priority under 35 USC 119 from Japanese Patent Application No. 2011-206130 filed on Sep. 21, 2011, the disclosure of which is incorporated by reference herein.

### BACKGROUND

1. Technical Field

The present invention relates to a radiation detection element, a radiographic image detection panel and a radiographic imaging device, and in particular, relates to a radiation detection element, a radiographic image detection panel and a radiographic imaging device that are applied to X-ray imaging of breasts (mammography).

2. Related Art

In the medical field, an FPD (Flat Panel Detector), in which an X-ray sensitive layer is disposed on a TFT (Thin Film Transistor) active matrix substrate and that can convert X-ray information directly into digital data, is used as the radiation detection element of a radiographic imaging device. In the radiation detection element, for example, plural scan lines and plural signal lines are disposed so as to intersect one another, and pixels are provided in the form of a matrix in correspondence with the respective intersection portions of the scan lines and signal lines. Further, these plural scan lines and plural signal lines are connected to external circuits (e.g., amp ICs or gate ICs) at the peripheral portion of the radiation detection element.

On the other hand, decreasing the pixel size of the radiation detection element is effective in improving the resolution of the FPD. In order to realize improvements in the resolution and sensitivity, a light detecting device is proposed in which the arrangement of the pixels is offset by half of a pitch in the X and Y directions, and interpolation processing among pixels is carried out on the basis of generated image information (see, for example, Japanese Patent Application Laid-Open (JP-A) No. 2003-255049). Further, there has also been proposed a device that improves the resolution and maintains the S/N, and also aims for improvement in the utilization rate of light, by making the shape of the pixels be a hexagonal shape and arranging the pixels in a honeycomb form (see, for example, JP-A No. 2006-29839).

In an X-ray imaging device for the breast (a mammography machine), that serves as a device that carries out image capturing for the purpose of medical diagnosis and that carries out X-ray imaging of the breast of a subject (a patient) for, for example, early detection of breast cancer, there is the demand for capturing of images of the chest wall side of the subject as much as possible. Therefore, there is the problem that the aforementioned external circuits cannot be disposed at the chest wall side of the radiation detection element. Further, the main purpose of imaging of a breast at a mammography machine is to discover breast cancer, and there is the problem that connection between the external circuits and the radiation detection element becomes difficult merely when the pixel shape is simply made to be hexagonal or the pixel size is reduced in order to depict minute calcifications.

### SUMMARY

The present invention was made in consideration of the above-described problems, and an object thereof is to capture high-resolution images by a radiation detection element or the like, even at the end portions of the radiation detection element.

A first aspect of the present invention provides a radiation detection element including:

a radiation detection section that is formed from plural pixels that are the same size and are arrayed in a two-dimensional form while adjacent to one another, and that detects radiation that has passed through a region that is an object of imaging of a subject;

plural scan lines that transfer signals that carry out switching control of switching elements provided respectively at the plural pixels; and

plural data lines that are disposed so as to intersect the scan lines, and that transfer electric signals read-out by the switching elements,

wherein the radiation detection section is made into a shape in which a pair of opposing sides is longer than another pair of opposing sides, and each of the plural pixels is made into a shape that is short in a short side direction of the radiation detection section and long in a long side direction of the radiation detection section.

A second aspect of the present invention provides the radiation detection element of the first aspect, wherein:

each of the plural pixels is a rectangle; and

the plural pixels are disposed such that long sides of the pixels that are adjacent contact one another in the long side direction of the radiation detection section and short sides of the pixels that are adjacent contact one another in the short side direction of the radiation detection section.

A third aspect of the present invention provides the radiation detection element of the first aspect, wherein:

each of the plural pixels is a rectangle;

the plural pixels are arrayed such that long sides of the pixels that are adjacent contact one another in the long side direction of the radiation detection section and short sides of the pixels that are adjacent contact one another in the short side direction of the radiation detection section; and

plural pixel lines, which are formed from plural pixels in a direction parallel to a long side of the radiation detection section, are disposed such that the respective pixels of the pixel lines that are adjacent are arrayed alternately in a direction parallel to a short side of the radiation detection section and are offset from one another by one-half of an array pitch in the direction parallel to the long side of the radiation detection section.

A fourth aspect of the present invention provides the radiation detection element of the first aspect, wherein:

each of the plural pixels is a compressed hexagon in which, in a state in which arbitrary two opposing sides of six sides of a regular hexagon are parallel to the short side direction of the radiation detection section, the regular hexagon is squashed in the short side direction of the radiation detection section;

while sides of the compressed hexagonal pixels that are adjacent contact one another, longer pixel widths of the pixels are parallel to a long side of the radiation detection section and shorter pixel widths are in a direction parallel to a short side of the radiation detection section; and

plural pixel lines, which are formed from plural pixels in a direction parallel to the long side of the radiation detection section, are disposed such that the respective pixels of the pixel lines that are adjacent are arrayed alternately in the direction parallel to the short side of the radiation detection section and are offset from one another by one-half of an array pitch so as to correspond to between pixels that are adjacent.

A fifth aspect of the present invention provides the radiation detection element of the first aspect, wherein:

each of the plural pixels is a compressed hexagon in which, in a state in which arbitrary two opposing sides of six sides of a regular hexagon are parallel to the long side direction of the radiation detection section, the regular hexagon is squashed in the short side direction of the radiation detection section;

while sides of the compressed hexagonal pixels that are adjacent contact one another, longer pixel widths of the pixels are parallel to a long side of the radiation detection section and shorter pixel widths are in a direction parallel to a short side of the radiation detection section; and

plural pixel rows, which are formed from plural pixels in a direction parallel to the short side of the radiation detection section, are disposed such that the respective pixels of the pixel rows that are adjacent are arrayed alternately in a direction parallel to the long side of the radiation detection section and are offset from one another by one-half of an array pitch so as to correspond to between pixels that are adjacent.

A sixth aspect of the present invention provides the radiation detection element of the first aspect, wherein the region that is the object of imaging is a breast of the subject, and X-ray imaging of the breast is carried out by the radiation detection section with a direction, which is parallel to the short side direction of the radiation detection section, being a depthwise direction from a chest wall side of the subject to a nipple side of the breast.

A seventh aspect of the present invention provides the radiation detection element of the first aspect, wherein the radiation detection section includes a semiconductor film that receives irradiation of the radiation and generates charges, and the charges are accumulated in storage capacitors provided respectively at the plural pixels, and the charges accumulated in the storage capacitors are read-out by the switching elements.

An eighth aspect of the present invention provides the radiation detection element of the first aspect, wherein the radiation detection section includes a scintillator that converts the radiation that has been irradiated into visible light, and, after the converted visible light is converted into charges at a semiconductor layer, electric signals corresponding to the charges are outputted by the switching elements.

A ninth aspect of the present invention provides the radiation detection element of the first aspect, further including:

a first external circuit that, via the plural scan lines, carries out switching control of the switching elements provided respectively at the plural pixels; and

a second external circuit that carries out a predetermined signal processing on the electric signals transferred through the plural data lines,

wherein any one external circuit among the first external circuit and the second external circuit is disposed at two short sides of the radiation detection section, and the other external circuit is disposed at one long side among two long sides of the radiation detection section.

A tenth aspect of the present invention provides the radiation detection element of the ninth aspect, wherein the plural scan lines or the plural data lines are disposed alternately one-by-one between the radiation detection section and the first external circuits or second external circuits that are disposed at the two short sides of the radiation detection section.

An eleventh aspect of the present invention provides a radiographic image detection panel having a radiation detection element, the radiation detection element including:

a radiation detection section that is formed from plural pixels that are the same size and are arrayed in a two-dimensional form while adjacent to one another, and that detects radiation that has passed through a region that is an object of imaging of a subject;

plural scan lines that transfer signals that carry out switching control of switching elements provided respectively at the plural pixels; and

plural data lines that are disposed so as to intersect the scan lines, and that transfer electric signals read-out by the switching elements, wherein

the radiation detection section is made into a shape in which a pair of opposing sides is longer than another pair of opposing sides, and each of the plural pixels is made into a shape that is short in a short side direction of the radiation detection section and long in a long side direction of the radiation detection section.

A twelfth aspect of the present invention provides a radiographic image detection panel having a radiation detection element, the radiation detection element including:

a radiation detection section that is formed from plural pixels that are the same size and are arrayed in a two-dimensional form while adjacent to one another, and that detects radiation that has passed through a region that is an object of imaging of a subject;

a first external circuit that carries out switching control of switching elements provided respectively at the plural pixels;

plural scan lines that transfer signals for the switching control from the first external circuit;

plural data lines that are disposed so as to intersect the scan lines, and that transfer electric signals read-out by the switching elements; and

a second external circuit that carries out a predetermined signal processing on the electric signals transferred through the plural data lines, wherein

the radiation detection section is made into a shape in which a pair of opposing sides is longer than another pair of opposing sides, and each of the plural pixels is made into a shape that is short in a short side direction of the radiation detection section and long in a long side direction of the radiation detection section, and any one external circuit among the first external circuit and the second external circuit is disposed at two short sides of the radiation detection section, and the other external circuit is disposed at one long side among two long sides of the radiation detection section.

A thirteenth aspect of the present invention provides a radiographic imaging device that carries out pickup of radiographic images by a radiographic image detection panel according to the eleventh aspect.

A fourteenth aspect of the present invention provides a radiographic imaging device that carries out pickup of radiographic images by a radiographic image detection panel according to the twelfth aspect.

In this way, in accordance with the present invention, there is the excellent effect that, in a radiation detection element, a radiographic image detection panel and a radiographic imaging device, high-resolution images can be captured also at the end portion sides of the radiation detector, and, particularly in applications to mammography, X-rays can reach to as far as the root portion (the chest wall side) of the breast, and therefore, loss does not arise in a captured image.

### BRIEF DESCRIPTION OF THE DRAWINGS

Exemplary embodiments of the present invention will be described in detail based on the following figures wherein:

FIG. 1 is a drawing illustrating the structure of a radiographic imaging device relating to a first exemplary embodiment of the present invention;

FIG. 2 is a cross-sectional view illustrating the structure of a radiation detection element of the radiographic imaging device relating to the present exemplary embodiment;

FIG. 3 is a drawing illustrating the structure of a radiographic imaging device relating to a second exemplary embodiment of the present invention;

FIG. 4 is a drawing illustrating the structure of a radiographic imaging device relating to a third exemplary embodiment of the present invention;

FIG. 5 is a drawing illustrating the structure of a radiographic imaging device relating to a fourth exemplary embodiment of the present invention;

FIG. 6 is a drawing illustrating the structure of a radiographic imaging device relating to a modified example; and

FIG. 7 is a block diagram illustrating an example of an X-ray diagnostic device for breasts (a mammography machine).

### DETAILED DESCRIPTION

Exemplary embodiments of the present invention are described in detail hereinafter with reference to the drawings. Note that, in the following exemplary embodiments, a radiographic imaging device 100 is an FPD (Flat Panel Detector) for mammography that is an X-ray diagnostic device for breasts. Further, in the following exemplary embodiments, description is given of cases in which the present invention is applied to a direct-conversion-type radiation detection element that directly converts radiation into charges.

### First Exemplary Embodiment

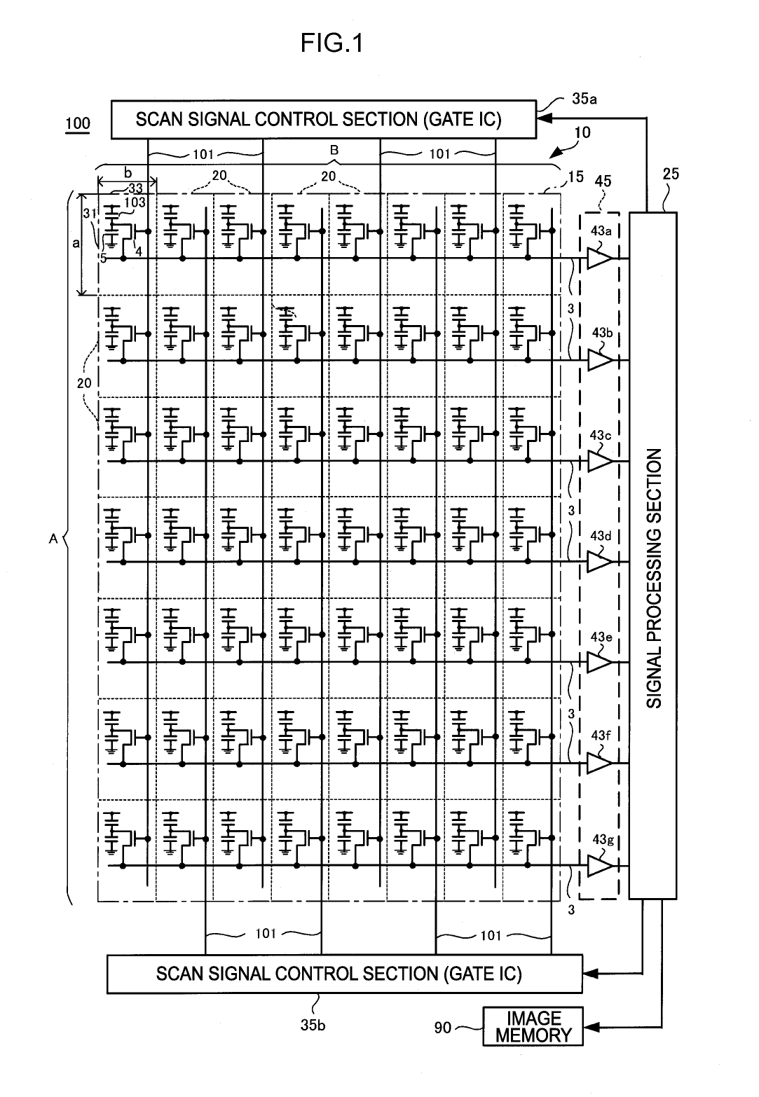

FIG. 1 illustrates the structure of the radiographic imaging device 100 relating to the first exemplary embodiment of the present invention. As illustrated in FIG. 1, the radiographic imaging device 100 relating to the present exemplary embodiment includes: a radiation detection element 10 that is formed from plural pixels 20; scan signal control sections (also called gate ICs) 35a, 35b that output, to scan lines, scan signals that are for turning on and off TFT switches within the respective plural pixels; a signal amplification section 45 that amplifies charge signals that have been transferred from data lines, in order to read-out charges accumulated in charge storage capacitors within the respective plural pixels; a signal processing section 25 that carries out signal processings such as converting the charge signals, that have been amplified at the signal amplification section 45, into digital image data by an unillustrated A/D converter, and the like; and an image memory 90 that stores the digital image data obtained by conversion at the signal processing section 25.

In the radiation detection element 10 of the radiographic imaging device 100 relating to the present exemplary embodiment, the plural pixels 20, that are each rectangular as seen in plan view, are disposed two-dimensionally in the form of a matrix while being adjacent to one another, and, on the whole, structure a pixel region 15 that is rectangular for example, as shown by long side A and short side B in FIG. 1. In order to improve the resolution of the radiographic imaging device 100, it is effective to reduce the size of the pixels structuring the radiation detection element 10. In particular, in a direct-conversion-type radiation detection element that uses selenium (Se) in the photoelectric conversion layer that absorbs radiation and converts the radiation into charges, reducing the pixel size as is contributes to an improvement in the resolution. Thus, in the present exemplary embodiment, the shape of each of the plural pixels 20 is made to be a rectangle in which a square is squashed in the left-right direction in FIG. 1 (the short side direction of the radiation detection element 10), i.e., a rectangle in which the length of the pixel 20 in the direction parallel to the chest wall side of the radiation detection element 10 is long, and that is short in the direction intersecting the direction parallel to the chest wall side.

Concretely, at each of the pixels 20, a dimension a of a side 31 at a long side A side of the radiation detection element 10 is 75 μm for example, and a dimension b of a side 33 at a short side B side is 50 μm for example, such that each pixel 20 is a shape (a rectangle) in which the side 31 is longer than the side 33. Further, here, the dimension a of the side 31 and the dimension b of the side 33 are in an integer ratio. Note that, although not illustrated, the pixel electrodes that collect the charges at the respective pixels 20 also have the same shapes as the respective pixels.

In X-ray diagnostic devices for breasts (mammography machines), the general method is, in a state in which a breast of a subject is placed on an imaging stand in which a radiographic image detector is built in, pressing the breast by a compression plate, irradiating radiation (X-rays) onto the breast from the compression plate side, and receiving the radiation transmitted through the breast at an image pickup medium, thereby capturing a radiographic image of the breast.

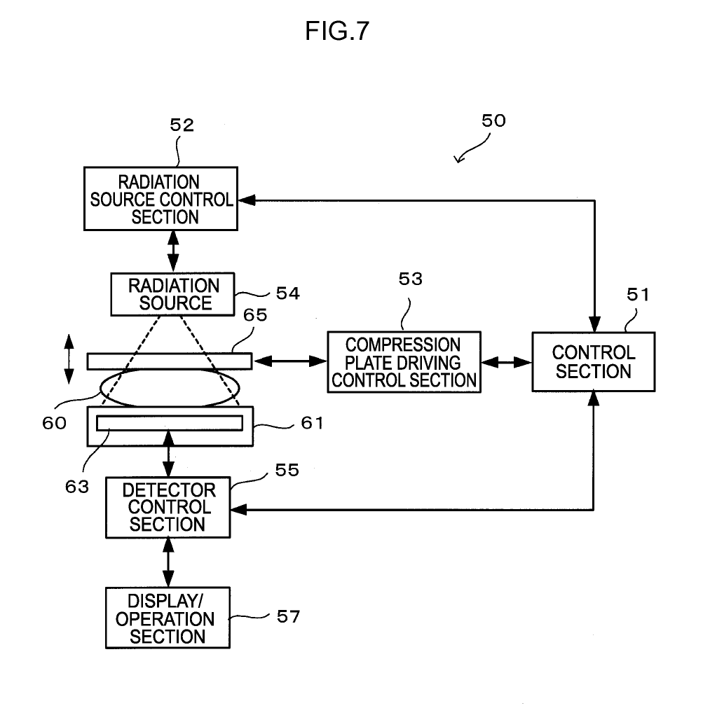

FIG. 7 is a block diagram showing an example of an X-ray diagnostic device for breasts (a mammography machine). A mammography machine 50 shown in FIG. 7 includes: a control section 51 that controls the device overall; a radiation source control section 52 that controls a radiation source 54; a compression plate driving control section 53 that displaces, in directions of the arrows in the drawing, a compression plate 65 that presses a breast 60 that is the object of imaging; and a detector control section 55 that controls a radiation detector 63, and transmits radiographic images detected at the radiation detector 63 to a display/operation section 57 or the like.

The compression plate 65 compresses the breast 60 against the imaging stand 61 and holds it thereat. Imaging conditions such as tube current, irradiation time and the like, which prescribe the dose of the radiation that is irradiated onto the breast 60 by the radiation source 54, are set at the radiation source control section 52. Further, imaging information such as the region to be imaged of the object of imaging (the breast 60), the imaging direction, and the like, can be set at the display/operation section 57, and the display/operation section 57 can display these imaging information.

In the case of a radiographic imaging device that is applied to mammography, there is the demand for picking-up images of minute calcifications in high detail. Although carrying out image pickup in high detail isotropically is preferable, effects are recognized even when image pickup in only one direction is carried out in high detail. The depthwise direction from the chest wall side to the nipple side of the breast that is the object of imaging can be picked-up in high detail, and, by making the side 33 in the aforementioned depthwise direction of each pixel 20 be shorter than side 31 in the direction parallel to the chest wall side, the depthwise direction from the chest wall side to the nipple side of the breast can be thoroughly imaged at a high resolution. Further, by setting the dimensions a and b of the sides to an integer ratio as described above, image conversion of the obtained image data into square matrix data is easy. Moreover, so-called binning processing in which, of the pixel information of the radiographic image, each two or more pixels thereof is gathered together as information of one pixel, i.e., plural pixels are read-in simultaneously, and the pixel values thereof are added together and processed, is easy.

In order to use the radiographic imaging device 100 relating to the present exemplary embodiment as a mammography machine, the radiographic imaging device 100 is structured such that no external circuits, such as scan signal control sections, signal amplification sections or the like, are provided whatsoever at one of the long sides (the one side A at the left side in FIG. 1) of the radiation detection element 10, and the long side A is the chest wall side of the subject. Namely, at the radiation detection element 10, when viewed in plan view, the side surface of one side (side A) that is a long side is made to be the chest wall end surface, and the radiation detection element 10 is used with this chest wall end surface abutting the chest wall of the subject. In this way, the detection region 15 can be set close to the end portion of the substrate of the radiation detection element 10 at the one side at which external circuits are not provided. As a result, the detection region 15 can be brought to the verge of the end portion of the substrate of the radiation detection element 10. The closer that the detection region 15 is to the substrate end portion, the more that portions near to the root of the breast can be imaged by the radiographic imaging device 100 that is used for mammography.

Each of the pixels 20 of the radiation detection element 10 is structured to include: a sensor portion 103 that receives radiation (X-rays) irradiated by an unillustrated radiation source (X-ray source) and generates charges; a charge storage capacitor 5 that accumulates the charges generated at the sensor portion 103; and a thin film transistor 4 (also called TFT switch) for reading-out the charges accumulated in the charge storage capacitor 5. Further, plural scan lines 101, which are signal paths for turning the TFT switches 4 of the individual pixels on and off, extend in a given direction (the vertical direction in FIG. 1) at the radiation detection element 10. Plural data lines 3, which are signal paths for reading-out the charges accumulated in the charge storage capacitors 5, extend in the lateral direction so as to be orthogonal to the scan lines 101. Here, the scan signal control sections 35a, 35b are disposed so as to sandwich the detection region 15 in from the vertical direction, and the plural scan lines 101 are connected alternately one-by-one to the scan signal control section 35a and the scan signal control section 35b.

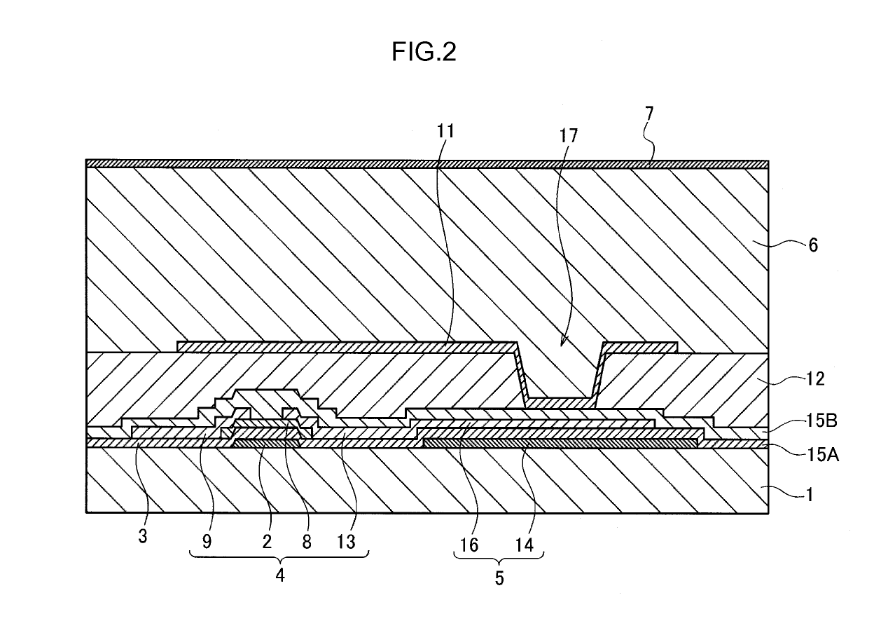

Moreover, as partially shown in FIG. 1, storage capacitor lines (also called common ground lines), that extend in the left-right direction in the drawing, are connected to ones of the electrodes of the charge storage capacitors 5. Note that a radiation-charge conversion material, such as amorphous selenium or the like, is used for the radiation detection element 10 as described later, and the radiation detection element 10 is a structure that converts radiation directly into charges. Further, a photoelectric conversion layer 6 (see FIG. 2) is provided so as to cover the charge storage capacitors 5 and the TFT switches 4. A semiconductor layer for example is used as the photoelectric conversion layer 6.

In the radiographic imaging device 100, the charge signals that are transferred from the individual data lines 3 are amplified at amplifiers (amps) 43a, 43b . . . 43g that are disposed within the signal amplification section 45 in correspondence with the individual data lines 3. The amplified charge signals are inputted to the signal processing section 25. At the signal processing section 25, in order to detect the electric signals inputted via the respective data lines 3, the charge signals that have been amplified as described above are held in sample hold circuits (not shown). The charge signals that are held in the individual sample hold circuits are inputted in order to a multiplexer (not shown), and thereafter, are converted into digital image data by an A/D converter (not shown).

Further, the signal processing section 25 outputs control signals, which express signal detection timings, to the scan signal control sections 35a, 35b. As a result, the scan signal control sections 35a, 35b receive the control signals from the signal processing section 25, and output signals for turning the TFT switches 4 on and off to the scan lines 101. Note that the signal processing section 25 has an unillustrated binning function section that integrates and reads-out signal charges of plural pixels for the aforementioned binning processing.

Moreover, as illustrated in FIG. 1, the image memory 90 is connected to the signal processing section 25. The digital image data outputted from the aforementioned A/D converter is stored in order in the image memory 90. The image memory 90 stores captured radiographic images as digital image data of several frames for example.

When capturing radiographic images at the radiographic imaging device 100 by using the radiation detection element 10, while radiation (X-rays) is being irradiated, OFF signals are outputted to the respective scan lines 101 and the respective TFT switches 4 are turned off, and the charges generated at a semiconductor layer that is described later are accumulated in the respective charge storage capacitors 5. Further, when reading-out an image, ON signals are outputted in order line-by-line to the respective scan lines 101 and the respective TFT switches 4 are turned on, and the charges accumulated in the respective charge storage capacitors 5 are read-out as electric signals. Due to the read-out electric signals being converted into digital data, a radiographic image of the region that is the object of pickup (in the case of a mammography machine, the breast of the subject) is obtained.

FIG. 2 is a cross-sectional view showing the structure of the radiation detection element 10. The radiation detection element 10 has a structure in which gate electrodes 2, the scan lines 101, and storage capacitor lower electrodes 14 are formed as a gate wiring layer on a substrate 1 that is insulating. The scan lines 101 are arranged one-by-one with respect to each pixel row that is formed from plural pixels (as illustrated in FIG. 1, the pixel row is structured by plural pixels that are continuous in the vertical direction), and are connected to the gate electrodes 2 that are formed at the respective pixels 20. This gate wiring layer for the gate electrodes 2 is formed by using, for example, a layered film of Al or Cu, or whose main component is Al or Cu.

Further, an insulating film 15A is formed on an entire of the gate wiring layer. The regions of the insulating film 15A, which are positioned on the gate electrodes 2, function as gate insulating films at the TFT switches 4. This insulating film 15A is formed from, for example, SiNₓ or the like, and is formed by CVD (Chemical Vapor Deposition) for example. Moreover, semiconductor active layers 8 are formed in shapes of islands above the gate electrodes 2 on the insulating film 15A. The semiconductor active layer 8 is the channel portion of the TFT switch 4, and is formed from, for example, an amorphous silicon film.

A source electrode 9 and a drain electrode 13 are formed at the upper layer of the gate electrode 2 and the like. Together with the source electrode 9 and the drain electrode 13, the data line 3 is formed in the wiring layer in which the source electrode 9 and the drain electrode 13 are formed. Further, storage capacitor upper electrodes 16 are formed on the insulating film 15A, at positions corresponding to the storage capacitor lower electrodes 14. The drain electrodes 13 are connected to the storage capacitor upper electrodes 16. The data lines 3 are connected to the source electrodes 9 that are formed at the pixels 20 of the respective pixel rows.

The wiring layer, which is shown in FIG. 2 and in which the source electrodes 9, the drain electrodes 13, the data lines 3 and the storage capacitor upper electrodes 16 are formed (also called source wiring layer), is formed by using a layered film of, for example, Al or Cu, or whose main component is Al or Cu. An impurity-added semiconductor layer (not illustrated), which is formed of impurity-added amorphous silicon or the like, is formed between, on the one hand, the source electrode 9 and the drain electrode 13, and, on the other hand, the semiconductor active layer 8. Note that, at the TFT switch 4, the source electrode 9 and the drain electrode 13 are opposite, in accordance with the polarity of the charges that are collected and accumulated by a lower electrode 11 that is described later.

A TFT protecting film layer 15B is formed over substantially the entire surface of the region that covers the source wiring layer and where the pixels are provided on the substrate 1 (substantially the entire region). The TFT protecting film layer 15B is formed of, for example, SiNₓ or the like, and by, for example, CVD. Further, a coated-type interlayer insulating film 12 is formed on the TFT protecting film layer 15B. The interlayer insulating film 12 is formed to a film thickness of 1 to 4 μm from a photosensitive organic material (e.g., a positive photosensitive acrylic resin: a material in which a naphthoquinonediazide positive photosensitive agent is mixed together with a base polymer formed from a copolymer of methacrylic acid and glycidyl methacrylate, or the like) having a low permittivity (dielectric constant εᵣ=2 to 4).

At the radiation detection element 10 of the radiographic imaging device 100 relating to the present exemplary embodiment, the capacity between metals that are disposed in the layer above and the layer below the interlayer insulating film 12 is kept low by the interlayer insulating film 12. Further, the aforementioned material that forms the interlayer insulating film 12 generally also functions as a smoothing film, and also has the effect that the steps of the layer beneath are smoothed. In the radiation detection element 10, contact holes 17 are formed in positions, of the interlayer insulating film 12 and the TFT protecting film layer 15B, which oppose the storage capacitor upper electrodes 16.

As illustrated in FIG. 2, the lower electrode 11 of the sensor portion 103 is formed so as to cover the pixel region while filling-in the contact hole 17, on the interlayer insulating film 12 and at each of the pixels 20. This lower electrode 11 is formed from an amorphous, transparent, electrically-conductive oxide film (ITO), and is connected to the storage capacitor upper electrode 16 via the contact hole 17. As a result, the lower electrode 11 and the TFT switch 4 are electrically connected via the storage capacitor upper electrode 16.

The photoelectric conversion layer 6 is formed uniformly on the lower electrodes 11 over substantially the entire surface of the pixel region where the pixels 20 are provided on the substrate 1. Due to radiation such as X-rays or the like being irradiated, the photoelectric conversion layer 6 generate charges (electrons-holes) at the interior thereof. Namely, the photoelectric conversion layer 6 is electrically conductive, and is for converting the image information carried by the radiation into charge information, and is formed from, for example, amorphous a-Se (amorphous selenium) whose main component is selenium and that has a film thickness of 100 to 1000 μm. Here, main component means having a content of greater than or equal to 50%. An upper electrode 7 is formed on the photoelectric conversion layer 6. The upper electrode 7 is connected to an unillustrated bias power source, and bias voltage (e.g., several kV) is applied thereto from the bias power source. The above-described plural scan lines 101, plural data lines 3 and switching elements 4 are disposed at the lower layer side of the sensor portions 103 that are formed from the photoelectric conversion layer 6.

Operation of the radiographic imaging device 100 relating to the present exemplary embodiment is described next. In the state in which bias voltage is applied between the above-described upper electrode 7 and storage capacitor lower electrode 14, when X-rays are irradiated on the photoelectric conversion layer 6, charges (electron-hole pairs) are generated within the photoelectric conversion layer 6. Because the photoelectric conversion layer 6 and the charge storage capacitors 5 are structured so as to be electrically connected in series, the electrons generated within the photoelectric conversion layer 6 move to the + (plus) electrode side, and the positive holes move to the − (minus) electrode side. At the time of image detection, OFF signals (0 V) are outputted to all of the scan lines 101 from the scan signal control sections 35a, 35b, and negative bias is applied to the gate electrodes 2 of the TFT switches 4. Due thereto, the respective TFT switches 4 are held in OFF states. As a result, the electrons generated within the photoelectric conversion layer 6 are collected by the lower electrodes 11 and are accumulated in the charge storage capacitors 5. Because the photoelectric conversion layer 6 generates charge amounts that correspond to the irradiated radiation, charges, which correspond to the image information carried by the radiation, are accumulated in the charge storage capacitors 5 of the respective pixels. Note that, in relation to the fact that the aforementioned voltage of several kV is applied between the upper electrodes 7 and the storage capacitor lower electrodes 14, the charge storage capacitors 5 must be made to be large as compared with the capacitors that are formed at the photoelectric conversion layer 6.

On the other hand, at the time of reading-out an image, ON signals are outputted from the scan signal control sections 35a, 35b to the respective scan lines 101 one-by-one and in order, and ON signals (e.g., signals whose voltage is +10 to 20 V) are successively applied to the gate electrodes 2 of the TFT switches 4 via the scan lines 101. Due thereto, the TFT switches 4 of the respective pixels 20 of the respective pixel rows in the scan line direction are turned on in order and row-by-row, and electric signals corresponding to the charge amounts accumulated in the charge storage capacitors 5 of the respective pixels 20 flow-out row-by-row to the data lines 3. The electric signals that flow to the respective data lines 3 are amplified at the signal amplification section 45. Then, on the basis of the amplified electric signals, the signal processing section 25 detects the charge amounts, that are accumulated in the charge storage capacitors 5, as information of the pixels that structure the image of the region that is the object of image pickup. Due thereto, image information for the image, which is expressed by the radiation irradiated on the radiation detection element 10, can be obtained.

As described above, in accordance with the first exemplary embodiment, the radiographic imaging device that is applied to mammography is structured such that the respective shapes of the plural pixels that are arranged in the form of a matrix at a radiation detection element that is rectangular in plan view, are made to be pixel shapes (rectangles) that are longer in the direction parallel to the chest wall side than the depthwise direction that is the direction intersecting the direction parallel to the chest wall side, and no external circuits whatsoever are provided at one long side of the radiation detection element, and this long side is the chest wall side of the subject. By doing so, at the one side at which no external circuits are provided, the region to be detected of the object of imaging can be set close to the end portion of the substrate of the radiation detection element, and the problem of X-rays not reaching the root of the breast in mammographic applications and loss arising in a captured image does not arise.

Further, by making the shape of each pixel of the radiation detection element be a shape (a rectangle) that is long in the direction parallel to the chest wall side and short in the depthwise direction of the radiation detection element, the resolution in the depthwise direction that is effective in mammographic applications, and the resolution, can be improved, and simultaneously, the distance between the data lines and the signal amplification section is short. Therefore, the effects of noise on the minute signals that are the electric signals that flow through the data lines and correspond to the charge amounts accumulated in the charge storage capacitors within the pixels, can be reduced.

Moreover, at the radiation detection element, the plural rectangular pixels are disposed in a matrix form in two dimensions while being adjacent to one another. Therefore, binning processing, in which a predetermined plural number of pixels is read-in simultaneously and the pixel values thereof are added, also can be carried out easily.

### Second Exemplary Embodiment

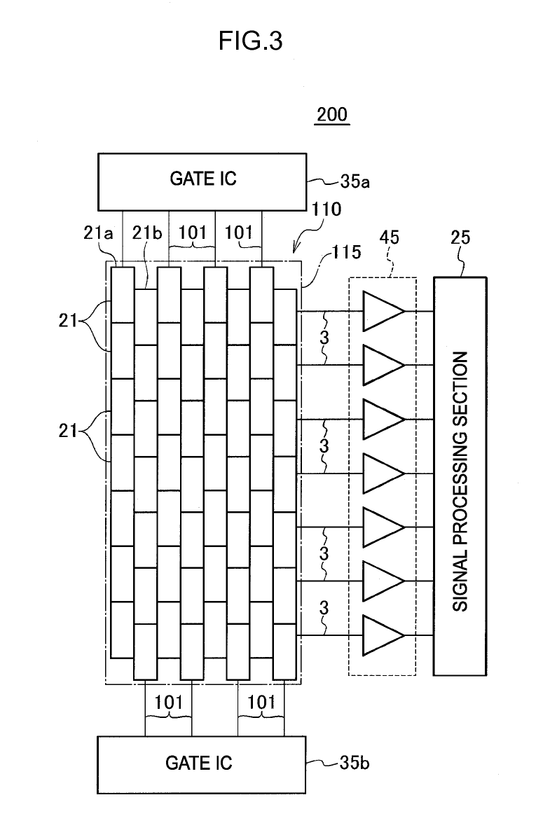

FIG. 3 illustrates the structure of a radiographic imaging device relating to a second exemplary embodiment of the present invention. In a radiographic imaging device 200 relating to the second exemplary embodiment shown in FIG. 3, structural elements that are the same as those of the radiographic imaging device 100 relating to the above-described first exemplary embodiment are denoted by the same reference numerals, and therefore, description thereof is appropriately omitted here.

As illustrated in FIG. 3, in a radiation detection element 110 of the radiographic imaging device 200 relating to the second exemplary embodiment, plural pixels 21 that are rectangular in plan view are disposed in a two dimensional form while being adjacent to one another. Further, at the radiation detection element 110, when the group of pixels that is disposed in the vertical direction in FIG. 3 is termed a “pixel line”, a first pixel line 21a, in which the rectangular pixels 21 of the same size are plurally arrayed in a predetermined direction (the vertical direction in FIG. 3), and a second pixel line 21b, in which the rectangular pixels 21 that are the same size as the pixels 21 of the first pixel line 21a are plurally arrayed in the vertical direction in the same way as at the first pixel line 21 a, are arrayed alternately in the direction intersecting the vertical direction (i.e., in the horizontal direction), and pixels that are adjacent in these pixel lines are disposed so as to be offset with respect to one another by one-half of the array pitch (one-half of a pixel). The same holds for the other pixel lines as well, and the pixels thereof are arrayed so as to be offset by one-half of a pixel from one another.

In the same way as the radiographic imaging device 100 relating to the above-described first exemplary embodiment, the radiographic imaging device 200 relating to the second exemplary embodiment is structured such that no external circuits, such as scan signal control sections, signal amplification sections or the like, are provided whatsoever at one of the long sides (the one side at the left side in FIG. 3) of the radiation detection element 110, and this long side is the chest wall side of the subject in applications to mammography. Further, at the radiation detection element 110, the pixels 21 are disposed such that the respective long sides of the rectangular pixels 21 are in the direction parallel to the chest wall side, and the short sides are in the depthwise direction.

The scan signal control sections (gate ICs) 35a, 35b are disposed so as to sandwich-in, from the vertical direction, a detection region 115 of the radiation detection element 110 that is formed from the plural pixels 21 that are arranged in this way. The plural scan lines 101 are connected, alternately one-by-one, between the radiation detection element 110 and the scan signal control section 35a, and between the radiation detection element 110 and the scan signal control section 35b. Note that, in FIG. 3, the internal circuit structure of the pixels 21 at the radiation detection element 110, the connections between these internal circuit elements and the data lines 3 and the scan lines 101, and the like are basically the same as in the radiation detection element 100 of the radiographic imaging device 100 relating to the first exemplary embodiment shown in FIG. 1, and therefore, illustration and description thereof are omitted.

In accordance with the radiographic imaging device relating to the second exemplary embodiment, at the one side of the radiation detection element at which external circuits are not provided, the region to be detected of the object of imaging can be set close to the end portion of the substrate of the radiation detection element, and images can be captured to as far as the root of the breast in mammographic applications. In addition, at the plural pixel lines in which plural rectangular pixels are arrayed in a predetermined direction, the pixels are disposed so as to be offset with respect to one another by one-half of the array pitch (one-half of a pixel). Due thereto, when compared with a radiation detection element in which the pixels are disposed without the array pitch being offset, the sampling points change, and therefore, there is the feature that data interpolation of intermediate portions in the long side direction can be carried out accurately.

Further, the pixel shape is a shape (a rectangle) that is long in the direction parallel to the chest wall side of the radiation detection element and is short in the depthwise direction. Therefore, not only does the resolution in the depthwise direction improve, but also, the distance between the data lines and the signal amplification section is close and the data lines can be made shorter. Therefore, the effects of noise on the minute electric signals that flow through the data lines can be reduced.

### Third Exemplary Embodiment

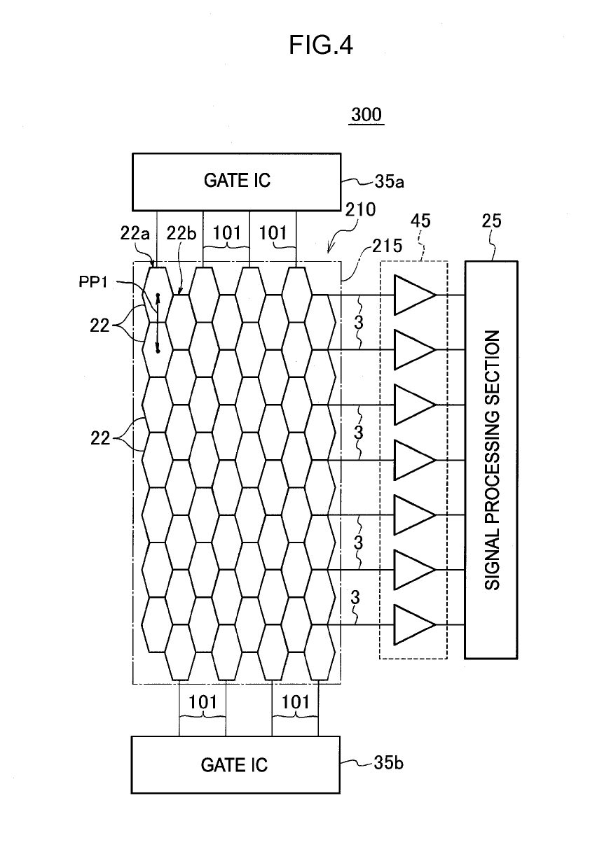

FIG. 4 illustrates the structure of a radiographic imaging device relating to a third exemplary embodiment of the present invention. Note that, in a radiographic imaging device 300 relating to the third exemplary embodiment shown in FIG. 4, structural elements that are the same as those of the radiographic imaging device 100 relating to the above-described first exemplary embodiment are denoted by the same reference numerals, and therefore, description thereof is appropriately omitted here.

As illustrated in FIG. 4, a radiation detection element 210 of the radiographic imaging device 300 relating to the third exemplary embodiment structures a pixel region 215 that overall is rectangular in plan view and in which plural pixels 22 that are shaped as compressed hexagons are disposed in a two-dimensional form while being adjacent to one another. Each of the pixels 22 has the shape of a compressed hexagon in which, while a regular hexagon is maintained with arbitrary two opposing sides of the six sides of the regular hexagon being in a state of being parallel to the short side direction of the radiation detection element 210, this regular hexagon is squashed in the short side direction of the radiation detection element 210 (in other words, compressed such that one diagonal line of the hexagon is shorter than the other two diagonal lines, and these other two diagonal lines are equal lengths). Further, at the radiation detection element 210, the pixels 22 are disposed such that the two sides, which are continuous in the same direction, of each of these compressed hexagonal pixels 22 are in the direction parallel to the chest wall side.

More specifically, at the radiation detection element 210, while the sides of the compressed hexagonal pixels 22 that are adjacent and the same size contact one another, the longer pixel widths of these pixels 22 are parallel to the long side of the radiation detection element 210, and the shorter pixel widths are arrayed in a direction parallel to the short side of the radiation detection element 210. Moreover, these pixels 22 are disposed such that, when the group of pixels that are continuous in the direction parallel to the long side of the radiation detection element 210 (the vertical direction in FIG. 4) is termed a “pixel line”, the pixels of a first pixel line 22a shown in FIG. 4, and the pixels of a second pixel line 22b that is adjacent in the vertical direction to this first pixel line 22a, are arrayed alternately in the direction parallel to the short side of the radiation detection element 210, and the pixels 22 of the second pixel line 22b are made to correspond to between adjacent pixels of the first pixel line 22a (in other words, the pixels 22 of the first pixel line 22a are made to correspond to between adjacent pixels of the second pixel line 22b), and pixels are offset from one another by one-half of the array pitch. The same holds for the other pixel lines as well, and the pixels thereof are in an arrayed state of being offset by one-half of a pixel from one another.

In the same way as the radiographic imaging device 100 relating to the above-described first exemplary embodiment, the radiographic imaging device 300 relating to the third exemplary embodiment also is structured such that no external circuits, such as scan signal control sections, signal amplification sections or the like, are provided whatsoever at one of the long sides (the one side at the left side in FIG. 4) of the radiation detection element 210, and this long side is the chest wall side of the subject in applications to mammography.

Further, in the radiographic imaging device 300 relating to the third exemplary embodiment, the scan signal control sections (gate ICs) 35a, 35b are disposed so as to sandwich-in, from the vertical direction, the detection region 215 that is formed from the plural pixels 22 that are arranged as described above. The plural scan lines 101 are connected alternately one-by-one between the radiation detection element 210 and the scan signal control section 35a, and between the radiation detection element 210 and the scan signal control section 35b. Note that, the internal circuit structure of the pixels 22 at the radiation detection element 210 in FIG. 4, the connections between these internal circuit elements and the data lines 3 and the scan lines 101, and the like are basically the same as in the radiation detection element 10 of the radiographic imaging device 100 relating to the first exemplary embodiment shown in FIG. 1, and therefore, illustration and description thereof are omitted.

In this way, in accordance with the radiographic imaging device relating to the third exemplary embodiment, at the one side of the radiation detection element at which external circuits are not provided, the region to be detected of the object of imaging can be set close to the end portion of the substrate of the radiation detection panel, and images can be captured to as far as the root of the breast in mammographic applications. In addition, by arranging the plural pixel lines, in which the plural compressed hexagonal pixels are arrayed in the direction parallel to the long side of the radiation detection element 210, so as to be offset with respect to one another by one-half of the array pitch (one-half of a pixel), most dense filling of pixels at the radiation detection element can be realized, and the resolution of the radiation detection element itself can be increased.

Further, because the pixel shape is a hexagonal shape that is compressed in the depthwise direction of the radiation detection element, the resolution in the depthwise direction improves. Further, the distance between the data lines and the signal amplification section is close and the data lines can be made to be shorter. Therefore, the effects of noise on the minute electric signals that flow through the data lines can be reduced.

Moreover, each of the pixels that structures the radiation detection element has the shape of a compressed hexagon in which, while a regular hexagon is maintained such that arbitrary two opposing sides of the six sides of the regular hexagon are parallel to the short side direction of the radiation detection element, this regular hexagon is squashed in the short side direction of the radiation detection element. Therefore, as compared with a radiographic imaging device relating to a fourth exemplary embodiment that is described hereafter, there can be made to be fewer indentations and protrusions of the pixels that are positioned at the chest wall side of the radiation detection element.

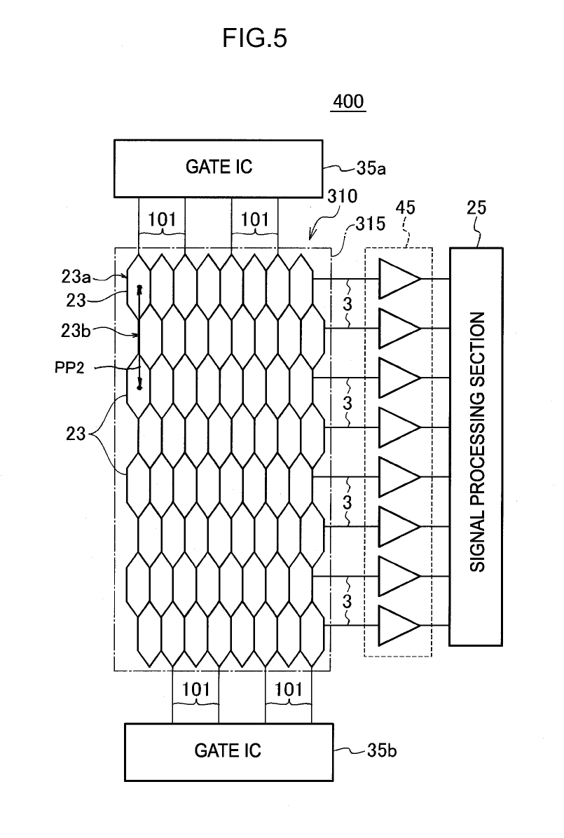

Still further, in the radiographic imaging device relating to the third exemplary embodiment, the array pitch (shown as PP1 in FIG. 4) of the pixels in the long side direction of the radiation detection element (in the case of mammography, the direction intersecting the depthwise direction from the chest wall side to the nipple side of the breast) is shorter than the array pitch (shown as PP2 in FIG. 5) of the pixels of the radiation detection element in the fourth exemplary embodiment that is described hereafter. Accordingly, the pixel arrangement in the radiographic imaging device relating to the third exemplary embodiment can ensure higher resolution in the long side direction of the pixel region than the pixel arrangement of the radiographic imaging device relating to the fourth exemplary embodiment that is described later. As a result, in mammography, highly-detailed radiographic images can be captured not only in the depthwise direction from the chest wall side to the nipple side of the breast, but also in the direction intersecting the depthwise direction.

### Fourth Exemplary Embodiment

FIG. 5 illustrates the structure of a radiographic imaging device relating to a fourth exemplary embodiment of the present invention. Note that, in a radiographic imaging device 400 relating to the fourth exemplary embodiment shown in FIG. 5, structural elements that are the same as those of the radiographic imaging device 100 relating to the above-described first exemplary embodiment are denoted by the same reference numerals, and therefore, description thereof is appropriately omitted here.

A radiation detection element 310 of the radiographic imaging device 400 relating to the fourth exemplary embodiment shown in FIG. 5 structures a pixel region 315 that overall is rectangular in plan view and in which plural pixels 23 that are shaped as compressed hexagons are disposed in a two-dimensional form while being adjacent to one another. Each of the pixels 23 has the shape of a compressed hexagon in which, while a regular hexagon is maintained such that arbitrary two opposing sides of the six sides of the regular hexagon are parallel to the long side direction of the radiation detection element 310, this regular hexagon is squashed in the short side direction of the radiation detection element 310 (which can also be called compressed such that, of the three diagonal lines that pass through the center of the pixel, one diagonal line is longer than the other two diagonal lines, and these other two diagonal lines are equal lengths). Further, at the radiation detection element 310, the pixels 23, which are shaped as compressed hexagons as described above, are disposed such that, of the respective six sides of the pixel 23, the two sides that oppose one another in the horizontal direction are in a direction parallel to the chest wall side.

To describe this point more specifically, at the radiation detection element 310, while the sides of the compressed hexagonal pixels 23 that are adjacent and the same size contact one another, the longer pixel widths of these pixels 23 are parallel to the long side of the radiation detection element 310, and the shorter pixel widths are arrayed in a direction parallel to the short side of the radiation detection element 310. Moreover, these pixels 23 are disposed such that, when a group of pixels that are continuous in the direction parallel to the short side of the radiation detection element 310 (the lateral direction in FIG. 5) is termed a “pixel row”, the pixels of a first pixel row 23a shown in FIG. 5, and the pixels of a second pixel row 23b that is adjacent in the lateral direction to this first pixel row 23a, are arrayed alternately in the direction parallel to the long side of the radiation detection element 310, and the pixels 23 of the second pixel row 23b are made to correspond to between adjacent pixels of the first pixel row 23a (in other words, the pixels 23 of the first pixel row 23a are made to correspond to between adjacent pixels of the second pixel row 23b), and pixels are offset from one another by one-half of the array pitch. The same holds for the other pixel rows as well, and the pixels thereof are in an arrayed state of being offset by one-half of a pixel from one another.

In the same way as the radiographic imaging device 100 relating to the above-described first exemplary embodiment, the radiographic imaging device 400 relating to the fourth exemplary embodiment also is structured such that no external circuits, such as scan signal control sections, signal amplification sections or the like, are provided whatsoever at one of the long sides (the one side at the left side in FIG. 5) of the radiation detection element 310. Further, this long side is the chest wall side of the subject in applications to mammography.

Further, in the radiographic imaging device 400 relating to the fourth exemplary embodiment as well, the scan signal control sections (gate ICs) 35a, 35b are disposed so as to sandwich-in, from the vertical direction, the detection region 315 that is formed from the plural pixels 23, and the plural scan lines 101 are connected alternately one-by-one to the scan signal control section 35a and the scan signal control section 35b. Note that, the internal circuit structure of the pixels 23 at the radiation detection element 310 in FIG. 5, the connections between these internal circuit elements and the data lines 3 and the scan lines 101, and the like are basically the same as in the radiation detection element 10 of the radiographic imaging device 100 relating to the first exemplary embodiment shown in FIG. 1, and therefore, illustration and description thereof are omitted.

As described above, in the radiographic imaging device relating to the fourth exemplary embodiment, at the one side of the radiation detection element at which external circuits are not provided, the region to be detected of the object of imaging can be set close to the end portion of the substrate of the radiation detection panel, and images can be captured to as far as the root of the breast in mammographic applications.

Further, because the pixel shapes are hexagonal shapes that are compressed in the depthwise direction of the panel, the resolution in the depthwise direction improves. In addition, because the distance between the data lines and the signal amplification section is close, as a result, the data lines can be made shorter, and the effects of noise on the minute electric signals that flow through the data lines can be reduced.

Note that the present invention is not limited to the above-described exemplary embodiments, and can be changed in various ways. In the above-described first through fourth exemplary embodiments, the scan signal control sections are disposed at the short sides of the radiation detection element (the detection region) so as to sandwich the radiation detection element in from the vertical direction. However, the present invention is not limited to the above-described first through fourth exemplary embodiments, provided that it is structured such that, in applications to mammography, no external circuits whatsoever are provided at one of the long sides of the radiation detection element and that long side is the chest wall side of the subject, and that the object of improving the resolution in the depthwise direction from the chest wall side to the nipple side of the breast can be achieved.

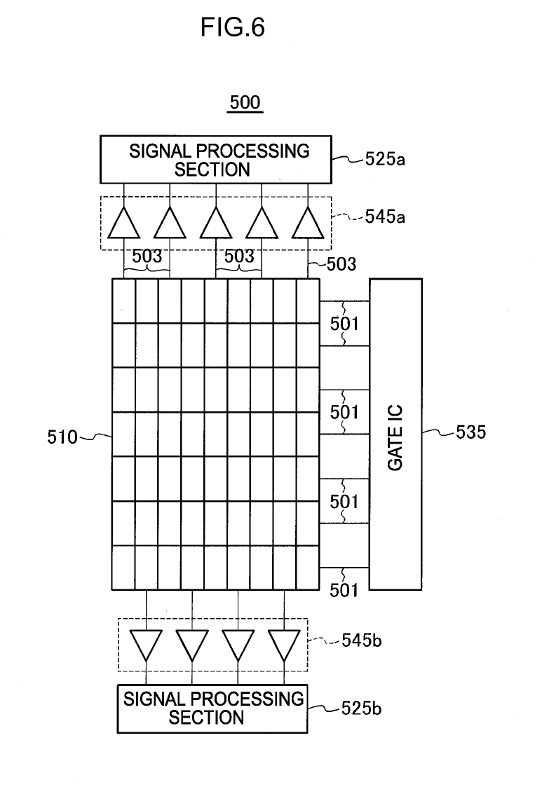

For example, as illustrated in FIG. 6, the respective shapes of the plural pixels that structure a radiation detection element 510 of a radiographic imaging device 500 are made to be shapes (rectangles) that are longer in the direction parallel to the chest wall side (the one side that is the left side in FIG. 6) than in the depthwise direction, and no external circuits are provided at the chest wall side of the radiation detection element 510, and this long side is the chest wall side of the subject. Further, the present invention may be structured such that signal amplification sections 545a, 545b and signal processing sections 525a, 525b are disposed so as to nip the radiation detection element 510 in from the vertical directions, and a scan signal control section (gate IC) 535 is disposed at the other long side of the radiation detection element 510.

Note that, here, data lines 503 from the radiation detection element 510 to the signal amplification section 545a, and the data lines 503 from the radiation detection element 510 to the signal amplification section 545b, are disposed alternately one-by-one.

In this way, in the radiographic imaging device shown in FIG. 6, the detection region can be brought right up to the end portion of the substrate of the radiation detection element 510. Therefore, in mammographic applications, the closer that the detection region of the radiation detection element 510 is to the substrate end portion, the closer to the root of the breast that the radiographic imaging device 500 can capture images.

Further, although the radiation detection element of a radiographic imaging device is described in the above exemplary embodiments, the range of application of the radiation detection element is not limited to this. For example, this radiation detection element may be applied to a radiographic image detection panel, or can be applied as well to a radiographic imaging device that uses, in image pickup, a radiographic image detection panel that has this radiation detection element.

Moreover, the above exemplary embodiments describe cases in which the present invention is applied to a direct-conversion-type radiation detection element. However, the present invention can be applied to an indirect-conversion-type radiation detection element that is provided with a scintillator that converts irradiated radiation into visible light, and in which, after the converted visible light is converted into charges at a semiconductor layer such as a photodiode or the like, electric signals corresponding to the charges are outputted by switching elements.

Still further, although the shape of the radiation detection section is rectangular in the above-described exemplary embodiments, the present invention is not limited to this. For example, the shape of the radiation detection section can be made to be a shape in which a pair of opposing sides is longer than the other pair of opposing sides, or in other words, a quadrilateral having a pair of opposing long sides and a pair of opposing short sides, or a trapezoid.

What is claimed is:

1. A radiation detection element comprising:
a radiation detection section that is formed from a plurality of pixels that are the same size and are arrayed in a two-dimensional form while adjacent to one another, and that detects radiation that has passed through a region that is an object of imaging of a subject;
a plurality of scan lines that transfer signals that carry out switching control of switching elements provided respectively at the plurality of pixels; and
a plurality of data lines that are disposed so as to intersect the scan lines, and that transfer electric signals read-out by the switching elements,
wherein the radiation detection section is made into a shape in which a pair of opposing sides is longer than another pair of opposing sides, and each of the plurality of pixels is made into a shape that is short in a short side direction of the radiation detection section and long in a long side direction of the radiation detection section.

2. The radiation detection element of claim 1, wherein:
each of the plurality of pixels is a rectangle; and
the plurality of pixels are disposed such that long sides of the pixels that are adjacent contact one another in the long side direction of the radiation detection section and short sides of the pixels that are adjacent contact one another in the short side direction of the radiation detection section.

3. The radiation detection element of claim 1, wherein:
each of the plurality of pixels is a rectangle;
the plurality of pixels are arrayed such that long sides of the pixels that are adjacent contact one another in the long side direction of the radiation detection section and short sides of the pixels that are adjacent contact one another in the short side direction of the radiation detection section; and
a plurality of pixel lines, which are formed from a plurality of pixels in a direction parallel to a long side of the radiation detection section, are disposed such that the respective pixels of the pixel lines that are adjacent are arrayed alternately in a direction parallel to a short side of the radiation detection section and are offset from one another by one-half of an array pitch in the direction parallel to the long side of the radiation detection section.

4. The radiation detection element of claim 1, wherein:
each of the plurality of pixels is a compressed hexagon in which, in a state in which arbitrary two opposing sides of six sides of a regular hexagon are parallel to the short side direction of the radiation detection section, the regular hexagon is squashed in the short side direction of the radiation detection section;
while sides of the compressed hexagonal pixels that are adjacent contact one another, longer pixel widths of the pixels are parallel to a long side of the radiation detection section and shorter pixel widths are in a direction parallel to a short side of the radiation detection section; and
a plurality of pixel lines, which are formed from a plurality of pixels in a direction parallel to the long side of the radiation detection section, are disposed such that the respective pixels of the pixel lines that are adjacent are arrayed alternately in the direction parallel to the short side of the radiation detection section and are offset from one another by one-half of an array pitch so as to correspond to between pixels that are adjacent.

5. The radiation detection element of claim 1, wherein:
each of the plurality of pixels is a compressed hexagon in which, in a state in which arbitrary two opposing sides of six sides of a regular hexagon are parallel to the long side direction of the radiation detection section, the regular hexagon is squashed in the short side direction of the radiation detection section;
while sides of the compressed hexagonal pixels that are adjacent contact one another, longer pixel widths of the pixels are parallel to a long side of the radiation detection section and shorter pixel widths are in a direction parallel to a short side of the radiation detection section; and
a plurality of pixel rows, which are formed from a plurality of pixels in a direction parallel to the short side of the radiation detection section, are disposed such that the respective pixels of the pixel rows that are adjacent are arrayed alternately in a direction parallel to the long side of the radiation detection section and are offset from one another by one-half of an array pitch so as to correspond to between pixels that are adjacent.

6. The radiation detection element of claim 1, wherein the region that is the object of imaging is a breast of the subject, and X-ray imaging of the breast is carried out by the radiation detection section with a direction, which is parallel to the short side direction of the radiation detection section, being a depthwise direction from a chest wall side of the subject to a nipple side of the breast.

7. The radiation detection element of claim 1, wherein the radiation detection section comprises a semiconductor film that receives irradiation of the radiation and generates charges, and the charges are accumulated in storage capacitors provided respectively at the plurality of pixels, and the charges accumulated in the storage capacitors are read-out by the switching elements.

8. The radiation detection element of claim 1, wherein the radiation detection section comprises a scintillator that converts the radiation that has been irradiated into visible light, and, after the converted visible light is converted into charges at a semiconductor layer, electric signals corresponding to the charges are outputted by the switching elements.

9. The radiation detection element of claim 1, further comprising:
a first external circuit that, via the plurality of scan lines, carries out switching control of the switching elements provided respectively at the plurality of pixels; and
a second external circuit that carries out a predetermined signal processing on the electric signals transferred through the plurality of data lines,
wherein any one external circuit among the first external circuit and the second external circuit is disposed at two short sides of the radiation detection section, and the other external circuit is disposed at one long side among two long sides of the radiation detection section.

10. The radiation detection element of claim 9, wherein the plurality of scan lines or the plurality of data lines are disposed alternately one-by-one between the radiation detection section and the first external circuits or second external circuits that are disposed at the two short sides of the radiation detection section.

11. A radiographic image detection panel having a radiation detection element, the radiation detection element comprising:
a radiation detection section that is formed from a plurality of pixels that are the same size and are arrayed in a two-dimensional form while adjacent to one another, and that detects radiation that has passed through a region that is an object of imaging of a subject;
a plurality of scan lines that transfer signals that carry out switching control of switching elements provided respectively at the plurality of pixels; and
a plurality of data lines that are disposed so as to intersect the scan lines, and that transfer electric signals read-out by the switching elements, wherein
the radiation detection section is made into a shape in which a pair of opposing sides is longer than another pair of opposing sides, and each of the plurality of pixels is made into a shape that is short in a short side direction of the radiation detection section and long in a long side direction of the radiation detection section.

12. A radiographic image detection panel having a radiation detection element, the radiation detection element comprising:
a radiation detection section that is formed from a plurality of pixels that are the same size and are arrayed in a two-dimensional form while adjacent to one another, and that detects radiation that has passed through a region that is an object of imaging of a subject;
a first external circuit that carries out switching control of switching elements provided respectively at the plurality of pixels;
a plurality of scan lines that transfer signals for the switching control from the first external circuit;
a plurality of data lines that are disposed so as to intersect the scan lines, and that transfer electric signals read-out by the switching elements; and
a second external circuit that carries out a predetermined signal processing on the electric signals transferred through the plurality of data lines, wherein
the radiation detection section is made into a shape in which a pair of opposing sides is longer than another pair of opposing sides, and each of the plurality of pixels is made into a shape that is short in a short side direction of the radiation detection section and long in a long side direction of the radiation detection section, and any one external circuit among the first external circuit and the second external circuit is disposed at two short sides of the radiation detection section, and the other external circuit is disposed at one long side among two long sides of the radiation detection section.

13. A radiographic imaging device that carries out pickup of radiographic images by a radiographic image detection panel according to claim 11.

14. A radiographic imaging device that carries out pickup of radiographic images by a radiographic image detection panel according to claim 12.
